(19) United States
(12) Patent Application Publication
(10) Pub. No.: US 2012/0004648 A1
(43) Pub. Date: Jan. 5, 2012

(54) SURGICAL INSTRUMENT

(76) Inventors: Seung Wook Choi, Gyeonggi-do (KR); Jong Seok Won, Gyeonggi-do (KR); Hyung Ho Kim, Gyeonggi-do (KR)

(21) Appl. No.: 13/254,679

(22) PCT Filed: Mar. 3, 2010
(86) PCT No.: PCT/KR2010/001314

§ 371 Date: Sep. 2, 2011

(30) Foreign Application Priority Data
Mar. 6, 2009 (KR) 10-2009-0019072

(51) Int. Cl. A61B 17/00

(52) U.S. Cl. 606/1

(57) ABSTRACT

A surgical instrument is disclosed that includes: a handle; a shaft coupled to the handle and extending in one direction; a bending part formed on a portion of the shaft; a driving part mounted on the handle and configured to generate a particular driving power; and a power transmission mechanism configured to transmit the driving power generated by the driving part to the bending part such that the bending part is bent. By mounting a driving part onto the surgical instrument and enabling the shaft to bend by a simple manipulation on a controller, it is possible to readily change the direction of the effector even when holding the instrument in one hand. Also, since the shaft is bent while the instrument is held, the reaction force applied on the effector when changing the direction of the effect can be felt by the user, providing a “haptic feedback”.

### CROSS-REFERENCE TO RELATED APPLICATIONS

This application is the National Phase of PCT/KR2010/001314 filed on Mar. 3, 2010, which claims priority under 35 U.S.C. 119(a) to Patent Application No. 10-2009-0019072 filed in the Republic of Korea on Mar. 6, 2009, all of which are hereby expressly incorporated by reference into the present application.

### BACKGROUND

The present invention relates to a surgical instrument.

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open so that an internal organ, etc., may be treated, reconstructed, or excised, can incur problems of blood loss, side effects, pain, scars, etc.

Thus, a popular alternative is “laparoscopic surgery” or “minimal invasive surgery”, which involves perforating a small insertion hole in the skin, instead of making an incision, and inserting an endoscope, laparoscope, surgical instrument, microscope for microsurgery, etc., through the insertion hole.

As illustrated in FIG. 1, a conventional surgical instrument 10 used for such laparoscopic surgery may be structured to have an effector 16 coupled to the end of a shaft 14 extending from a handle 12. When the direction of the effector 16 has to be changed during surgery, the conventional instrument is manipulated by a person manually releasing a locking device 18, manually operating a manipulator 26 to bend a portion 20 of the shaft like a joint, and then locking the locking device 18 again.

This conventional method of manually manipulation, however, requires manual manipulation of the locking device 18 and the manipulator 26 during surgery, and thus requires both hands to manipulate the instrument 10. As such, the surgical instrument 10 cannot be manipulated, if there are surgical tools held in both hands already.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the invention is to provide a surgical instrument with which the shaft can be bent by a simple manipulation on a controller to readily change the direction of the effector.

One aspect of the invention provides a surgical instrument that includes: a handle; a shaft coupled to the handle and extending in one direction; a bending part formed on a portion of the shaft; a driving part mounted on the handle and configured to generate a particular driving power; and a power transmission mechanism configured to transmit the driving power generated by the driving part to the bending part such that the bending part is bent.

An effector can further be included that is coupled to one end of the shaft and operated to perform an action required for surgery according to a user manipulation on the handle. In this case, a rotation manipulator can further be included that is rotatably mounted on the handle and connected to the effector to enable the effector to rotate about an extending direction of the shaft. Also, the effector can include a pair of jaws that engage each other, and a grip manipulator can be coupled to the handle and connected to the pair of jaws to enable the pair of jaws to open and close.

The bending part can be formed adjacent to the effector, which may change direction according to the curving of the bending part. In this case, the bending part can include a snake type joint.

The driving part can include: a motor part; a power source part configured to supply power required for driving the motor part; and a controller configured to control a driving of the motor part. The power source part can include a battery carried within the handle. The motor part can be positioned exterior to the handle and be connected to the handle by a driving power transmission mechanism. The controller can include a direction manipulator configured to control a driving of the motor part to bend the bending part in correspondence with a manipulation direction.

The driving part can include a hold capability for driving the motor part such that, if the direction manipulator is manipulated in a particular direction, the bending part remains in a bent state in correspondence with the manipulation. Also, the driving part can further include a release manipulator configured to control a driving of the motor part to restore the bending part to its initial state.

In this case, the direction manipulator can be manipulated in two or more directions, and the motor part can include a first motor and a second motor, the first motor driven in correspondence with a first manipulation direction of the direction manipulator, and the second motor driven in correspondence with a second manipulation direction of the direction manipulator. The bending part can be bent in a first direction according to a driving of the first motor and be bent in a second direction according to a driving of the second motor.

The power transmission mechanism can include a first wire, which connects the first motor with two points on the bending part facing each other along the first direction, and a second wire, which connects the second motor with two points on the bending part facing each other along the second direction. Also, the power transmission mechanism can further include a reaction force isolator part that is interposed between the first and second wires and the bending part and is configured to block a reaction force, by which the bending part tends toward returning to an original position, from being transmitted to the first and second motors. The reaction force isolator part can include a worm gear. The worm gear can include a worm and a worm wheel mating with the worm, where the worm can be separated from the worm wheel in correspondence with a manipulation on a release manipulator equipped on the driving part.

The shaft can be detachably coupled to the handle, and the power transmission mechanism can include a first power transmission mechanism built into the shaft and a second power transmission mechanism built into the handle, where the first power transmission mechanism and the second power transmission mechanism can be connected to each other when the shaft is coupled to the handle.

By mounting a driving part, e.g. a motor, etc., onto the surgical instrument and enabling the shaft to bend by a simple manipulation on a controller, e.g. a joystick, etc., a preferred embodiment of the invention makes it possible to readily change the direction of the effector even when holding the instrument in one hand. Also, since the shaft is bent while the instrument is held, a significant portion of the reaction force that is applied on the effector when changing the direction of the effect can be felt by the user, providing a “haptic feedback”.

Additional aspects and advantages of the present invention will be set forth in part in the description which follows, and in part will be obvious from the description, or may be learned by practice of the invention.

### BRIEF DESCRIPTION OF THE DRAWINGS

FIG. 1 is a perspective view illustrating an instrument for robot surgery according to the related art.

FIG. 2 is a side-elevational view conceptually illustrating a surgical instrument according to an embodiment of the invention.

FIG. 3 is a plan view illustrating the curving of a bending part according to an embodiment of the invention.

FIG. 4 is a magnified view of portion “A” in FIG. 3.

FIG. 5 illustrates a driving part according to an embodiment of the invention.

FIG. 6 illustrates a reaction force isolator part according to an embodiment of the invention.

FIG. 7 illustrates the operation of a release manipulator according to an embodiment of the invention.

FIG. 8 illustrates the detachable coupling of a shaft and a handle according to an embodiment of the invention.

### DETAILED DESCRIPTION

As the present invention allows for various changes and numerous embodiments, particular embodiments will be illustrated in the drawings and described in detail. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another. For example, a first component can be referred to as a second component, and likewise a second component can be referred to as a first component, without departing from the scope of the invention. When a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Also, in providing descriptions referring to the accompanying drawings, those components that are the same or are in correspondence are rendered the same reference numeral regardless of the figure number, and redundant descriptions are omitted. In the written description, certain detailed explanations of related art are omitted, when it is deemed that they may unnecessarily obscure the essence of the present invention.

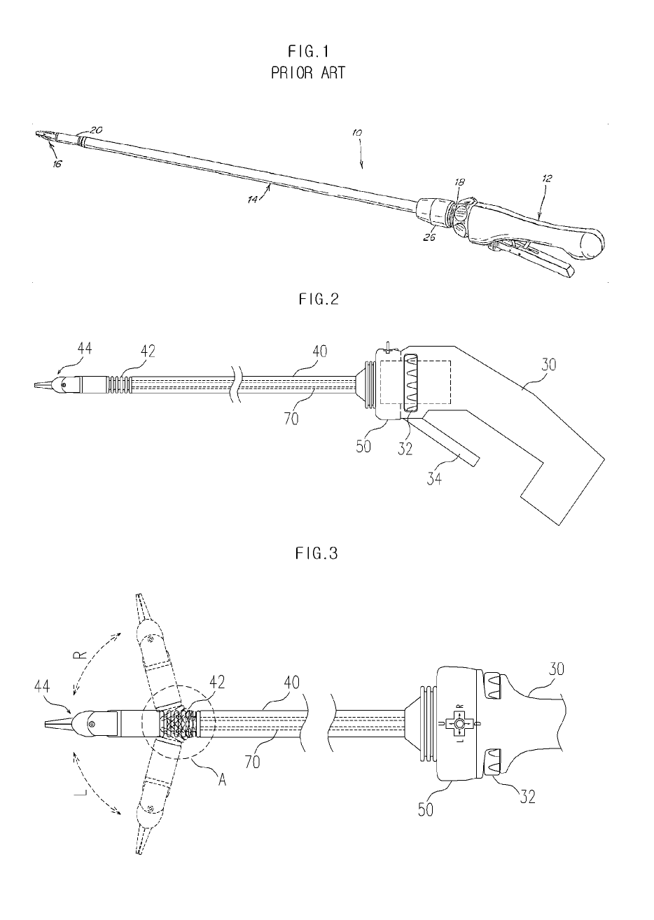

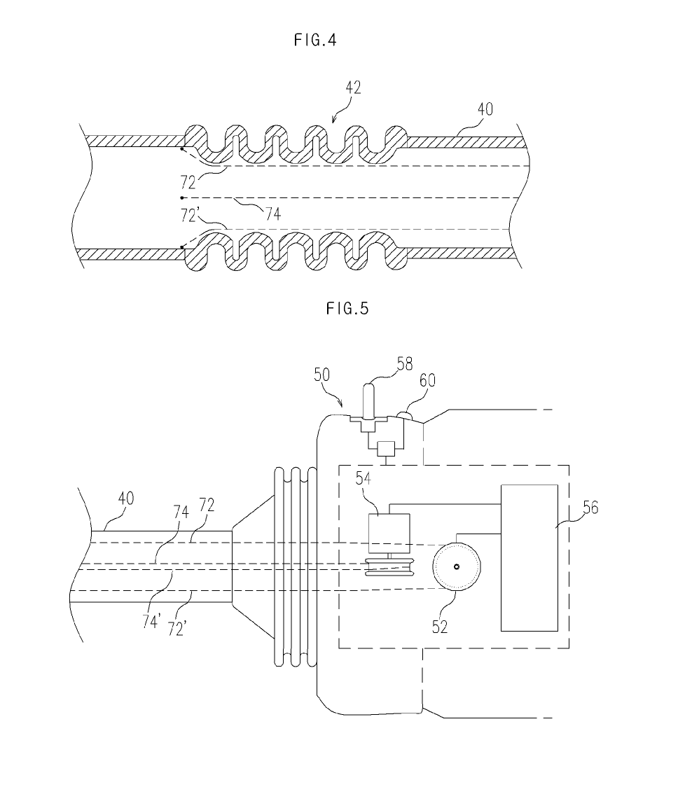

FIG. 2 is a side-elevational view conceptually illustrating a surgical instrument according to an embodiment of the invention, FIG. 3 is a plan view illustrating the curving of a bending part according to an embodiment of the invention, and FIG. 4 is a magnified view of portion “A” in FIG. 3. Illustrated in FIG. 2 to FIG. 4 are a handle 30, a rotation manipulator 32, a grip manipulator 34, a shaft 40, a bending part 42, an effector 44, a driving part 50, a direction manipulator 58, a power transmission mechanism 70, a first wire 72, and a second wire 74.

With this embodiment, instead of manually implementing a manipulation for bending the shaft 40 of the surgical instrument to change the direction of the effector 44 coupled to the end of the shaft 40 as in the related art, the bending may be performed automatically by using the power of a motor, etc. Thus, by simply manipulating a switch while holding the instrument, the direction of the effector 44 can be changed easily, and during this process, the user can be made able to sense the reaction force applied on the front end of the effector 44, i.e. the user can be provided with haptic feedback.

An instrument according to this embodiment relates to a so-called “motorized handheld instrument”. As illustrated in FIG. 2, a bending part 42 may be formed on the end of the shaft 40, and as illustrated in FIG. 3, the bending part 42 may be curved freely using motor power. Thus, for an instrument manipulated manually, the shaft 40 can be made to bend by using the force of a motor, etc., so that the end portion of the instrument can move up, down, left, and right.

As illustrated in FIG. 2, an instrument according to this embodiment has a basic structure of a shaft 40 extending from a manipulating handle 30. A bending part 42 capable of bending freely may be formed on the end portion of the shaft 40, a driving part 50 for generating a driving power that curves the bending part 42 may be mounted on the handle 30, and the driving part 50 and bending part 42 may be connected by a power transmission mechanism 70, so that the bending part 42 may curve in a desired direction according to the driving of the driving part 50.

A forceps-like effector 44 made of a pair of jaws can be coupled to the end of the shaft 40, and as the user holds and manipulates the handle 30, the effector 44 may perform various actions required for surgery, such as cutting, gripping, rotating, etc. To this end, various manipulators corresponding to the actions of the effector 44 can be coupled onto the handle 30, such as a rotation manipulator 32 that enables the effector 44 to rotate about the extending direction of the shaft 40, a grip manipulator 34 that enables the pair of jaws to open and close and perform a gripping action, and so on.

Accordingly, each manipulator and the effector 44 can be connected in various ways to implement various actions on the effector 44. For example, as illustrated in FIG. 2, the rotation manipulator 32 rotatably coupled to the handle 30 may be connected with the shaft 40, and may thereby enable the shaft 40 as well as the effector 44 connected to its far end to rotate according to the rotation of the rotation manipulator 32, and the grip manipulator 34 coupled to the handle 30 in the form of a lever may be connected by wires with the pair of jaws, so that when the grip manipulator 34 is pulled on, the tensional force may be transmitted via the wires, to enable the effector 44 to perform a gripping action. Various other connecting methods can be applied according to the operating method of the effector 44 and the operating method of each manipulator.

The bending part 42 may be formed on a portion of the shaft 40 to serve as a joint at which the shaft 40 curves in a certain direction. In the embodiment illustrated in FIG. 2, the bending part 42 is formed on an end portion of the shaft 40, i.e. at a portion adjacent to the effector 44.

As the driving part 50 generates and transmits a driving power to cause the bending part 42 to curve, as described above, the portion of the shaft 40 beyond the bending part 42, i.e. the portion to which the effector 44 is coupled, may move in a particular direction, and thus the direction in which the effector 44 is facing may be changed by the curving of the bending part 42.

That is, not only is it possible to manipulate an instrument according to this embodiment such that the effector 44 performs various actions (cutting, gripping, rotating, etc.) required for surgery, it is also possible to change the direction in which the effector 44 is facing, so that various surgical manipulations can be performed on a desired surgical site without having to change the direction of the entire instrument. Furthermore, even in situations where the direction of the instrument cannot be changed, the effector 44 can be made to change direction in performing surgery by bending the shaft 40.

To thus curve a portion of the shaft 40, a bending part 42 according to this embodiment can be implemented by combining various devices such as hinges, pivots, bellows, etc., or be implemented in a so-called “snake type” form, having multiple flexible joints installed continuously as illustrated in FIG. 4.

With the bending part 42 having a snake type form, it is possible to secure wires onto its inner walls and provide bending in a desired direction by transmitting tensional forces through the wires, as will be described later. For example, if four wires corresponding to the up, down, left, and right directions are secured onto the inner walls of the bending part 42, pulling on the left wire and loosening the right wire can cause the bending part 42 to bend left, due to the tension of the wire.

A description has been provided above on the overall structure of a surgical instrument according to this embodiment. However, it is not absolutely necessary that a motor be used for a driving part 50 according to this embodiment; various mechanisms for generating driving power can be used to curve the bending part 42. Also, it is not absolutely necessary that the driving power generated and transmitted from the driving part 50 be used for curving the bending part 42; the driving power can be utilized as the driving power for various manipulations such as operating the effector 44, etc.

Furthermore, an instrument according to this embodiment is not necessarily limited to a manual type in which the user holds the instrument in one hand; a setup can be implemented in which the handle portion 30 is mounted on a surgical robot arm and is operated by driving power transmitted from the robotic arm. A more detailed description will now be provided as follows on the operation of each part of the instrument.

FIG. 5 illustrates a driving part according to an embodiment of the invention. Illustrated in FIG. 5 are a shaft 40, a driving part 50, a first motor 52, a second motor 54, a power source part 56, a direction manipulator 58, a release manipulator 60, a first wire 72, and a second wire 74.

FIG. 5 is used to illustrate an example in which a driving part 50 according to this embodiment is implemented by a pulley-coupling wires onto a pair of motors.

A driving part 50 according to this embodiment can be composed of a motor part, a power source part 56, and a controller that controls the driving of the motor part. In this embodiment, the motor may be used for only the curving manipulation of the bending part 42, and a motor can be used with a capacity tantamount to generating the required power. In this case, a motor can be used having a size that allows the motor to be carried within the handle 30.

However, it is not absolutely necessary that a motor part according to this embodiment be carried within the handle; it is also possible to install the motor part exterior to the handle and connect the motor part with the handle by a driving power transmission mechanism such as a “cable conduit”, etc., so as to supply driving power from the exterior.

The power source part 56 may be a component that supplies electrical power required for driving the motor, and can receive electrical power from the exterior or employ a battery, such as of a regular or rechargeable type, etc., carried within the handle in order to improve the portability of the instrument. As described above, a miniature motor can be used to generate a power sufficient for curving the bending part 42, and accordingly, the power source part 56 can also be designed with a small capacity, so that the driving part 50 can thus be composed with low cost.

The controller may be a part that controls the driving of the motor part. With the present embodiment, a direction manipulator 58 such as a miniature joystick or direction keys can be installed as a controller on the handle portion 30 in a manner corresponding to the curving direction of the bending part 42, so that the curving direction of the bending part 42 can be controlled more intuitively. For example, if the bending part 42 is formed to bend in the up, down, left, and right directions, a joystick, etc., can be installed that is manipulated in the up, down, left, and right directions, such that the manipulation direction of the direction manipulator 58 matches the curving direction of the bending part 42, allowing the user to intuitively manipulate the direction manipulator 58 and curve the bending part 42.

As illustrated in FIG. 3, manipulating the direction manipulator 58 to the right (R) may correspondingly curve the bending part 42 to the right (R), while manipulating the direction manipulator 58 to the left (L) may correspondingly curve the bending part 42 to the left (L).

In order that the manipulation of the direction manipulator 58 in an up, down, left, or right direction may drive a motor to curve the bending part 42, a motor part according to this embodiment can employ a pair of motors 52, 54 as illustrated in FIG. 5.

That is, by making provisions such that the first motor 52 is driven according to an up and down manipulation of the direction manipulator 58, the second motor 54 is driven according to a left and right manipulation of the direction manipulator 58, the bending part 42 is moved up and down according to the driving of the first motor 52, and the bending part 42 is moved left and right according to the driving of the second motor 54, the manipulation direction of the direction manipulator 58 and the curving direction of the bending part 42 can be matched.

However, it is not absolutely necessary that the manipulation direction of the direction manipulator 58 and the curving direction of the bending part 42 according to this embodiment be set to up and down directions and left and right directions. Obviously, the movements of the direction manipulator 58 and the bending part 42 can be matched with the direction manipulator 58 manipulated in any two direction, i.e. a first manipulation direction and a second manipulation direction, and the bending part 42 curved in a corresponding manner in a first direction and a second direction.

Each point of the first and second motors 52, 54 and the bending part 42 can be connected by a power transmission mechanism 70 such as a wire, etc. For example, the first motor 52 can be connected to two points on the bending part 42 by a first wire 72, and the second motor 54 can be connected to two points on the bending part 42 by a second wire 74.

If the first motor 52 corresponds to curving along the up and down directions, and the second motor 54 corresponds to curving along the left and right directions, as in the example described above, then the first wire 72 connected to the first motor 52 can be connected to two points, an upper and a lower position, of the bending part 42, while the second wire 74 connected to the second motor 54 can be connected to two points, a left and a right position, of the bending part 42. Furthermore, if the first motor 52 corresponds to curving in a first direction, and the second motor 54 corresponds to curving in a second direction, then the first wire 72 can be connected to two points on the bending part 42 facing each other along the first direction, while the second wire 74 can be connected to two points on the bending part 42 facing each other along the second direction.

In addition to the direction manipulator 58, a driving part 50 according to the present embodiment can include a “hold” capability and a “release” capability.

The hold capability may keep the bending part 42 in a curved state if the direction manipulator 58 remains untouched. When the direction manipulator 58 is manipulated in a particular direction, the hold capability may lock the driving part 50 in place such that the bending part 42 maintains its bent position in correspondence with the manipulation.

Various methods can be used to implement this hold capability, such as by having the electrical power continuously supplied to the motor part when the direction manipulator 58 is in a manipulated state in a particular direction so that the motors 52, 54 are not operated by external forces and remain still, by choosing a motor having a sufficiently large cogging torque such that the reaction force resulting from the curving of the bending part 42 does not conversely operate the motors 52, 54 in order that the motors 52, 54 may not be operated by external forces besides the manipulation of the direction manipulator 58, by installing a stopper, etc., on the direction manipulator 58 so that the direction manipulator 58 is mechanically locked after being manipulated in a particular direction, or by installing a separate hold switch which may be pressed to lock the manipulation of the direction manipulator 58 and maintain the bent state of the bending part 42.

Correspondingly to the hold capability, a release capability can be implemented. The release capability may cause the shaft 40, which has been bent to a particular state, to automatically return to its original position when a release manipulator 60 such as a release button, etc., is manipulated. The release manipulator 60 may serve to drive the motor part to restore the bending part 42 to its initial state.

The release manipulator 60 can be implemented various ways. For example, the release manipulator 60 can be implemented in the form of a separate button. Alternatively, a press capability can be added to a joystick, so that the joystick may operate as the direction manipulator 58 when the joystick is manipulated up, down, left, or right, and as the release manipulator 60 when the joystick is pressed.

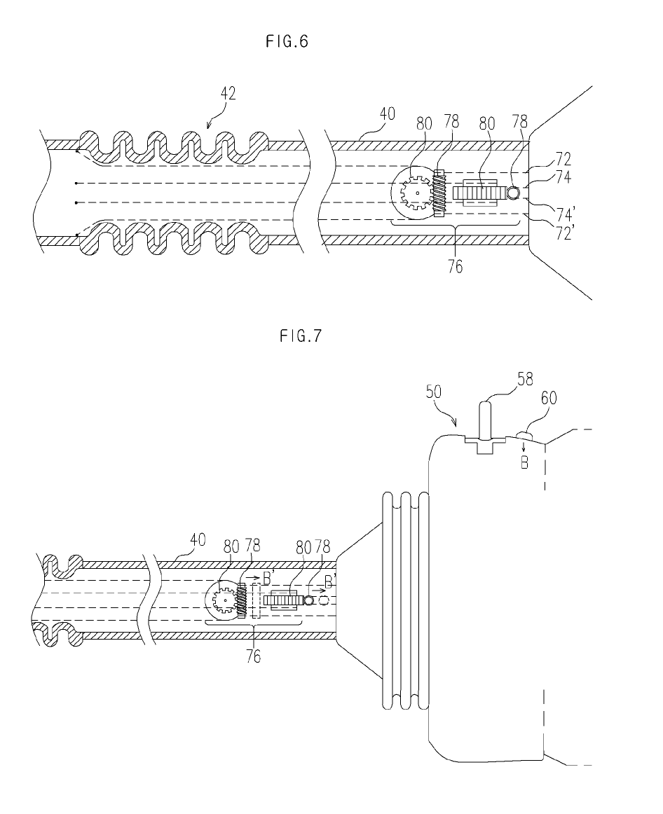

FIG. 6 illustrates a reaction force isolator part according to an embodiment of the invention, and FIG. 7 illustrates the operation of a release manipulator according to an embodiment of the invention. Illustrated in FIG. 6 and FIG. 7 are a shaft 40, a bending part 42, a driving part 50, a direction manipulator 58, a release manipulator 60, first wires 72, second wires 74, a reaction force isolator part 76, a worm 78, and a worm wheel 80.

This embodiment is illustrated for an example in which a reaction force isolator part 76 is installed in the middle of the power transmission mechanism 70, so that when power is generated and transmitted from the driving part 50 to curve the bending part 42, the reaction force resulting from the curving of the bending part 42 does not conversely act on the motor and cause damage to or malfunctioning of the motor.

That is, in connecting the driving part 50 and the bending part 42 with wires, a gear such as a worm gear 78, 80 can be placed in-between. Thus, the driving power may be transmitted from the driving part 50 to the bending part 42 (forward direction), but conversely a reaction force due to bending from the bending part 42 to the driving part 50 (reverse direction) may be isolated such that it is not directly transmitted to the motor.

By placing the reaction force isolator part 76, such as a worm gear 78, 80, etc., between the first and second wires 72, 74 and the bending part 42 as illustrated in FIG. 6, reaction forces of the bending part 42 tending to return to its original position can be prevented from being directly transmitted to the first and second motors 52, 54 and incurring unnecessary loads.

In this case, the forces generated and transmitted from the motor may not be applied directly on the bending part 42. Rather, the motor may rotate the worm 78, and the worm wheel 80 mating with the worm 78 may rotate correspondingly, so that the tensional forces may be applied to each point of the bending part 42. In this way, a reaction force resulting from the curving of the bending part 42 can be prevented from being conversely transmitted directly to the motors.

As described above, it is possible to implement a “release capability” for restoring the curved shaft 40 to its original position, by installing a release manipulator 60 on the driving part 50 and driving the motor part such that the bending part 42 bent into a particular state is restored to its initial state. Not only is it possible to electronically restore the shaft 40 according to a manipulation of the release button in this manner, it is also possible to implement the release capability by mechanically separating the reaction force isolator part 76.

That is, in cases where a worm gear having a worm 78 and a mating worm wheel 80 is used for the reaction force isolator part 76, manipulating the release manipulator 60 (see B of FIG. 7) can cause the worm 78 to be mechanically separated from the worm wheel 80 and be drawn back (see B′ of FIG. 7), so that the reaction force resulting from curving the bending part 42, i.e. the elastic restoring force of the bending part 42, may return the shaft 40 to its original position.

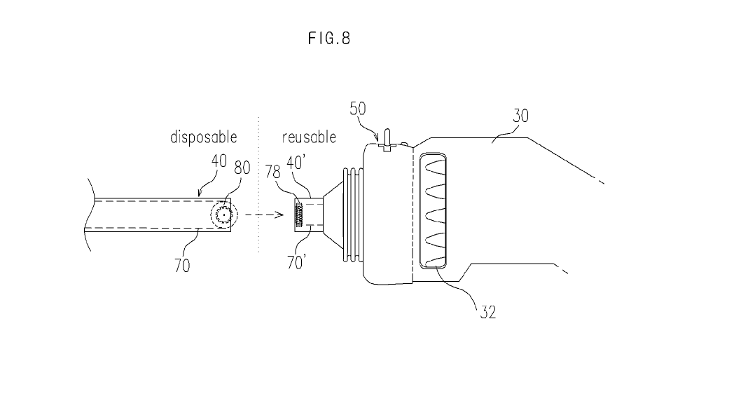

FIG. 8 illustrates the detachable coupling of a shaft and a handle according to an embodiment of the invention. Illustrated in FIG. 8 are a handle 30, a rotation manipulator 32, a shaft 40, a driving part 50, a power transmission mechanism 70, a worm 78, and a worm wheel 80.

An instrument according to this embodiment can be manufactured to have a structure in which the shaft portion 40 is disposable and the handle portion 30 is reusable. That is, the shaft portion 40 and the handle portion 30, 40′ can each be manufactured separately but in a detachably coupling structure, so that the shaft portion 40 may be discarded after use, and a new shaft may be fastened again to the handle 30 for reuse as necessary.

In this case, a power transmission mechanism 70 according to this embodiment may be separated into a first power transmission mechanism 70 built into the shaft side 40 and a second power transmission mechanism 70′ built into the handle side 30. By forming coupling devices that engage each other on the end portions of the separated power transmission mechanisms 70, 70′, respectively, the first power transmission mechanism 70 and second power transmission mechanism 70′ can be connected with each other during the coupling of the shaft 40 to the handle 30.

For example, a protrusion can be formed at the end portion of a wire built into the shaft side 40, and an indentation into which the protrusion may fit can be formed at the end portion of a wire built into the handle side 30 (40′), so that when coupling the shaft 40 onto the handle 30, the wires may be coupled and connected with each other.

Also, if a reaction force isolator part 76 such as a worm gear 78, 80, etc., is to be installed in the shaft 40, 40′ as described with reference to FIG. 6, the worm 78 can be installed in one side 40′ of the separated shaft and the worm wheel 80 mating with the worm 78 can be installed in the other side 40. Thus, during the process of separating and coupling the shaft 40, the worm gear can be mated, so that the power transmission mechanisms 70, 70′ may be connected naturally.

However, it is not absolutely necessary that in an instrument according to this embodiment the shaft has to be separated at a portion adjacent to the handle; it is also possible to manufacture the shaft to have a structure that it is separated in its middle portion, such that one portion is connected to the handle and the other portion serves as the shaft tip, with the two portions detachably coupling with each other. The shaft tip can be disposable, while the handle and the shaft portion connected to the handle can be reusable.

While the present invention has been described with reference to particular embodiments, it will be appreciated by those skilled in the art that various changes and modifications can be made without departing from the spirit and scope of the present invention, as defined by the claims appended below.

1. A surgical instrument comprising:
a handle;
a shaft coupled to the handle and extending in one direction;
a bending part formed on a portion of the shaft;
a driving part mounted on the handle, comprising a motor part, a power source part configured to supply power required for driving the motor part, and a controller configured to control a driving of the motor part, and configured to generate a particular driving power; and
a power transmission mechanism configured to transmit the driving power generated by the driving part to the bending part such that the bending part is bent,
wherein the controller comprises a direction manipulator configured to control a driving of the motor part to bend the bending part in correspondence with a manipulation direction.

2. The surgical instrument of claim 1, wherein the bending part comprises a snake type joint.

3. The surgical instrument of claim 1, wherein the shaft has an effector coupled to one end thereof, the effector operated to perform an action required for surgery according to a user manipulation on the handle, and
the bending part is formed adjacent to the effector, the effector configured to change direction according to a curving of the bending part.

4. The surgical instrument of claim 3, wherein the effector comprises a pair of jaws engaging each other, and
the handle has a rotation manipulator and a grip manipulator coupled thereto, the rotation manipulator connected to the effector to enable the effector to rotate about an extending direction of the shaft, the grip manipulator connected to the pair of jaws to enable the pair of jaws to open and close.

5. (canceled)

6. The surgical instrument of claim 1, wherein the power source part comprises a battery carried within the handle.

7. The surgical instrument of claim 1, wherein the motor part is positioned exterior to the handle and is connected to the handle by a driving power transmission mechanism.

8. (canceled)

9. The surgical instrument of claim 1, wherein the driving part includes a hold capability for driving the motor part such that, if the direction manipulator is manipulated in a particular direction, the bending part remains in a bent state in correspondence with the manipulation.

10. The surgical instrument of claim 1, wherein the driving part further comprises a release manipulator configured to control a driving of the motor part to restore the bending part to an initial state.

11. The surgical instrument of claim 1, wherein the direction manipulator is manipulated in two or more directions, and
the motor part comprises a first motor and a second motor, the first motor driven in correspondence with a first manipulation direction of the direction manipulator, the second motor driven in correspondence with a second manipulation direction of the direction manipulator.

12. The surgical instrument of claim 11, wherein the bending part is bent in a first direction according to a driving of the first motor and is bent in a second direction according to a driving of the second motor.

13. The surgical instrument of claim 12, wherein the power transmission mechanism comprises a first wire and a second wire, the first wire connecting the first motor with two points on the bending part facing each other along the first direction, the second wire connecting the second motor with two points on the bending part facing each other along the second direction.

14. The surgical instrument of claim 13, wherein the power transmission mechanism further comprises a reaction force isolator part interposed between the first wire and the bending part, the reaction force isolator part configured to block a reaction force, by which the bending part tends toward returning to an original position, from being transmitted to the first motor.

15. The surgical instrument of claim 14, wherein the reaction force isolator part comprises a worm gear.

16. The surgical instrument of claim 14, wherein the worm gear comprises a worm and a worm wheel mating with the worm, and
the worm is separated from the worm wheel in correspondence with a manipulation on a release manipulator equipped on the driving part.

17. The surgical instrument of claim 1, wherein the shaft is detachably coupled to the handle, the power transmission mechanism includes a first power transmission mechanism built into the shaft and a second power transmission mechanism built into the handle, and the first power transmission mechanism and the second power transmission mechanism are connected to each other when the shaft is coupled to the handle.
